(19) United States
(12) Patent Application Publication
(10) Pub. No.: US 2015/0276738 A1
(43) Pub. Date: Oct. 1, 2015

(54) ASSAY FOR ANALYSES OF RABIES VIRUS GLYCOPROTEIN

(71) Applicant: The USA, as represented by the Secretary, Dept. of Health & Human Service,Centers for Disease Contro, Rockville, MD (US)

(72) Inventors: Todd Gareth Smith, Decatur, GA (US); Charles Rupprecht, Atlanta, GA (US)

(21) Appl. No.: 14/434,910

(22) PCT Filed: Oct. 15, 2013
(86) PCT No.: PCT/US2013/064911

§ 371 Date: Apr. 10, 2015

Related U.S. Application Data
(60) Provisional application No. 61/713,130, filed on Oct. 12, 2012.

(51) Int. Cl. G01N 33/569; C12N 7/00; A61K 39/205

(52) U.S. Cl. CPC G01N 33/56983; A61K 39/205; C12N 7/00; G01N 2333/145; C12N 2760/20134; G06F 19/22

(57) ABSTRACT

Provided are processes of determining die immimogenicity of a rabies virus vaccine preparation that for the first tiine correlates well with in vivo results. The methods capitalize on an ECL assay for RABV G protein that is sensitive, reproducible, and can be used to quickly assess characteristics of new vaccine preparations.

### CROSS REFERENCE TO RELATED APPLICATIONS

This application depends from and claims priority to U.S. Provisional Application No. 61/713,130 filed Oct. 12, 2012, the entire contents of which are incorporated herein by reference.

### GOVERNMENT SPONSORSHIP

This invention was made by the Centers for Disease Control and Prevention, an agency of the United States Government.

### FIELD OF THE INVENTION

The present invention relates to detection of a virus. More particularly, the invention relates to detection of a virus of the Lyssavirus genus. Processes are provided that allow for the determination of a Lyssavirus or component thereof, specifically a rabies virus.

### BACKGROUND OF THE INVENTION

Rabies is an ancient disease with the earliest reports possibly dated to the Old World before 2300 B.C. Rabies remains a world health threat due to remaining lack of effective control measures in animal reservoir populations and a widespread lack of human access to vaccination. Today, more than 50,000 people annually die of rabies, particularly in Asia and Africa.

Lyssaviruses are RNA viruses with single-strand, negative-sense genomes responsible for rabies-like diseases in mammals. The Lyssavirus genus (family Rhabdoviridae) includes eleven recognized species. The type species Rabies virus (RABV) is distributed worldwide among mammalian reservoirs including carnivores and bats. There are many reported cases each year of transmission of RABV from animals to humans. To date, effective prophylaxis is able to prevent unwanted complications from RABV infection of humans. RABV, however, continues to present challenges for vaccine development due to neurotropism, immune evasion, antigenic diversity, lack of effective anti-viral drugs, and access to affordable modern biologics.

RABV vaccine potency testing is a confounding factor to current and future vaccine production. The NIH mouse inoculation test has been used over the past 50 years as a measure of RABV vaccine potency. The NIH vaccine potency test continues to present several issues for use and correlation with effective vaccination in humans including the unnatural routes of vaccination and challenge, the requirement of 154 mice per test, the wide confidence interval of results, and the associated time and costs of in vivo testing (1). Refinement and replacement of this historical measure continues to be an issue facing vaccine manufactures (4).

As such, new methods of determining the necessary RABV vaccine administration amounts, type of immunogen, immunogenicity, and effectiveness of vaccination are required.

### SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole.

Provided is a process of determining immunogenicity of a rabies virus vaccine. The processes of the invention can be used to screen for an active immunogen that will be sufficient to induce a protective immune response in a subject following administration to the subject. The processes of the invention provide the ability to quickly ascertain whether a potential or known protein will function as a robust immunogen without the need for extensive and time consuming in vivo testing.

A process of determining immunogenicity of a rabies virus vaccine includes interacting a test RABV glycoprotein with a first anti-glycoprotein antibody; binding the glycoprotein with a second anti-glycoprotein antibody simultaneous with the first anti-glycoprotein antibody, where the second glycoprotein antibody includes an electrochemiluminescent marker; and detecting the magnitude of signal produced by the second anti-glycoprotein antibody using said marker. The first anti-glycoprotein antibody is optionally a conformationally specific antibody that binds an epitope in antigenic site III of RABV glycoprotein. The process optionally further includes determining the immunogenicity of the test RABV glycoprotein as a rabies virus vaccine by the step of detecting. Some embodiments include the step of producing the vaccine or rejecting said vaccine for production where production is understood as manufacturing for subsequent administration. Optionally, producing the vaccine is performed when an ECL value (signal) in the process is or exceeds 4,400 in a sample with a protein concentration of 10 micrograms per milliliter. The processes optionally includes comparison to a glycoprotein potency standard curve. A glycoprotein potency standard curve is optionally established by interacting a plurality of known standard RABV glycoprotein concentrations with said an anti-glycoprotein antibody indistinguishable from said first anti-glycoprotein antibody; binding said standard glycoprotein with a detection anti-glycoprotein antibody indistinguishable from said second anti-glycoprotein antibody; detecting the quantity of said standard glycoprotein using a marker on said detection anti-glycoprotein antibody for each concentration to produce a standard signal; and constructing said standard curve using said signals. The electrochemiluminescent marker on said second anti-glycoprotein antibody optionally includes a SULFO-TAG NHS-ester. A process optionally includes comparing the quantity of said glycoprotein to a glycoprotein immunogenicity standard. Optionally, the second glycoprotein antibody binds a linear epitope on the glycoprotein. A process optionally further includes administering a therapeutically effective amount of a vaccine to a subject, where the vaccine includes a RABV glycoprotein determined to have sufficient immunogenicity in said process. The said therapeutically effective amount is optionally determined by the step of detecting. A RABV glycoprotein is optionally unlabeled or absent an immunoenhancing tag. In some embodiments the first antibody and said second antibody are compositionally identical. Optionally, the first anti-glycoprotein antibody and the second anti-glycoprotein antibody are both conformationally specific antibodies.

Also provided are processes of determining the immunogenicity of a rabies virus vaccine including interacting a test RABV glycoprotein with a first conformationally specific anti-glycoprotein antibody; binding the glycoprotein with a second anti-glycoprotein antibody simultaneous with the first anti-glycoprotein antibody, where the second glycoprotein antibody further includes an electrochemiluminescent marker; and detecting the magnitude of signal produced by the second anti-glycoprotein antibody using the marker where the immunogenicity is determined by the magnitude of the signal relative to a standard signal, a known signal, or a control. Optionally, the first anti-glycoprotein antibody, the second anti-glycoprotein antibody, or both are antibody 2-21-14. Optionally, the first anti-glycoprotein antibody binds an epitope in antigenic site III. A process optionally further includes determining sufficient immunogenicity of the test RABV glycoprotein as a rabies virus vaccine by the step of detecting, wherein the test RABV glycoprotein has an ECL value in process that is or exceeds 4.400 in a sample with a protein concentration of 10 micrograms per milliliter; and further comprising producing the vaccine comprising the test RABV glycoprotein. A process optionally further includes comparing the signal generated in said assay by said test RABV glycoprotein to a glycoprotein immunogenicity standard. Optionally, a therapeutically effective amount of said RABV glycoprotein is determined by said step of detecting, the process further comprising administering said therapeutically effective amount or greater of said RABV glycoprotein to a subject. Optionally, the first anti-glycoprotein antibody and the second anti-glycoprotein antibody are the same antibody.

Also provided are processes of determining the immunogenicity of a rabies virus vaccine including interacting a test RABV glycoprotein with antibody 2-21-14; binding the test RABV glycoprotein with a second anti-glycoprotein antibody simultaneous with the first anti-glycoprotein antibody, the second glycoprotein antibody including an electrochemiluminescent marker; and detecting the magnitude of signal produced by the second anti-glycoprotein antibody using the marker. Optionally, the second anti-glycoprotein antibody is antibody 2-21-14, antibody 62-80-6, or antibody 62-71-3. Optionally, the electrochemiluminescent marker includes a SULFO-TAG NHS-ester. The process optionally further includes producing test RABV glycoprotein as a vaccine or rejecting the RABV glycoprotein for production as a vaccine. The step of producing is optionally performed when the ECL value of the signal generated in the process is or exceeds 4,400 in a sample with a test RABV protein concentration of 10 micrograms per milliliter. A process optionally further includes administering a therapeutically effective amount of a vaccine including the test RABV glycoprotein to a subject.

### BRIEF DESCRIPTION OF THE DRAWINGS

FIG. 1 illustrates RABV G antigen captured and detected using the 2-21-14αRABV G MAb by ECL according to one embodiment of the invention where the ECL was measured for eight 5-fold serial dilutions of different rabies vaccine preparations, ECL counts were plotted against the total protein concentration on logarithmic scales, means of at least four statistical replicates from at least two biological replicates are shown with the symbols indicating as follows: purified RABV G ERA G lot 2005 (▪) Medicago® (*), ERA G lot 2012 (♦), RabAvert® (▪), RabAvert® depleted (). Imovax® (−), CVS G lot 2011 (×), ERA G lot 2010 (▴), Fraunhofer® (+), Imovax® depleted (♦), and Fraunhofer® Adjuvant ():

FIG. 2 illustrates the linear regression of the ECL counts plotted against the total protein concentration on a linear scale with exemplary linear regressions shown for RabAvert®, 4000 counts/(μg/ml) (dotted line); RabAvert® diluted, 3000 counts/(μg/ml) (dashed line); and RabAvert® depleted, 1000 counts/(μg/ml) (solid line); and

FIG. 3 illustrates the geometric mean rVNA titer measured 30 days after immunizing mice with selected rabies vaccines plotted against the log transformed ECL counts/(μg/ml).

### DETAILED DESCRIPTION OF EMBODIMENTS OF THE INVENTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the processes or kits are described as an order of individual steps or using specific materials, it is appreciated that described steps or materials may be interchangeable such that the description of the invention includes multiple parts or steps arranged in many ways as is readily appreciated by one of skill in the art.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Wild, D., The Immunoassay Handbook, 3rd Ed., Elsevier Science, 2005; Gosling, J. P., Immunoassays: A Practical Approach, Practical Approach Series, Oxford University Press, 2005; Antibody Engineering, Kontermann, R. and Dübel, S. (Eds.), Springer, 2001; Harlow, E. and Lane, D., Antibodies: A Laboratory Manual, Cold Spring Harbor Laboratory Press, 1988; Ausubel, F. et al., (Eds.), Short Protocols in Molecular Biology, Wiley, 2002; J. D. Pound (Ed.) Immunochemical Protocols, Methods in Molecular Biology, Humana Press; 2nd ed., 1998; B. K. C. Lo (Ed.), Antibody Engineering: Methods and Protocols, Methods in Molecular Biology, Humana Press, 2003; and Kohler, G. and Milstein, C., Nature, 256:495-497 (1975); the contents of each of which are incorporated herein by reference.

The present invention includes a process for selecting a vaccine for production or for predicting the immunogenicity of a RABV glycoprotein based immunogen. The process capitalizes on an unexpected relationship between the results of an inventive conformationally specific assay using electrochemiluminescent detection methodology and the traditional NIH test for determining which vaccine or antigen preparations will have sufficient immunogenicity to become valuable vaccines. As such, the invention has utility for the development of new diagnostics, therapeutics and prophylactic therapies for viral infection.

A process is provided that includes interacting a test RABV glycoprotein that can be used as an immunogen or a vaccine candidate with a first anti-glycoprotein antibody that is optionally conformationally specific, binding the glycoprotein with a second anti-glycoprotein antibody optionally simultaneous or sequentially with said first anti-glycoprotein antibody, wherein the second glycoprotein antibody includes an electrochemiluminescent marker; detecting the magnitude of signal produced by the second anti-glycoprotein antibody or the first anti-glycoprotein antibody using the marker; optionally comparing the quantity of the glycoprotein or magnitude of the signal to a glycoprotein immunogenicity standard; optionally determining the immunogenicity of said test RABV glycoprotein as a rabies virus vaccine by the step of detecting, and optionally producing a vaccine or optionally rejecting a vaccine for production based on the detecting.

The process in some embodiments capitalizes on immunosorbance of a conformationally specific antibody to a RABV glycoprotein. In some embodiments, the conformationally specific antibody is CDC mAb 2-21-14, which may be made publically available obtained from the Centers for Disease Control, Atlanta, Ga., or an antibody that interacts with an antigen that is identical to or overlaps with the epitope region(s) in the RABV glycoprotein and is specific for the conformation of the glycoprotein. Such conformationally specific antibodies will differentially bind glycoprotein that has sufficient antigenicity and the binding will be readily detectable by ECL processes. It is appreciated that prior processes such as known traditional ELISA assays using such conformationally specific antibodies as in this invention are unable to predict immunogenicity with sufficient confidence to be useful in moving an immunogen into production.

In some embodiments, an ECL tag used in a secondary antibody or directly labeled conformationally specific antibody is a SULFO-TAG NHS-ester, although it is appreciated that other tags known in the art may similarly be suitable.

A second antibody optionally recognizes a linear epitope in glycoprotein or recognizes the conformationally specific antibody with the proviso that the secondary antibody does not compete for the epitope of the primary antibody, optionally the conformationally specific antibody the like of CDC mAb 2-21-14. In some embodiments, a glycoprotein is optionally isolated with an antibody that is not conformationally specific, and a secondary antibody that does recognize a conformationally specific antibody is used for subsequent detection. In some embodiments, a conformationally specific antibody includes a label. In some embodiments, a linear epitope recognizing antibody includes a label. Optionally, both a conformationally specific and a linear epitope recognizing antibody include a label that may be the same or different. Known variations of immunosorbent assays with respect to detecting an antigen such as a sandwich assay, or using multiple secondary antibodies may be used with the present invention.

An antibody that is a conformationally specific antibody or recognizes a linear epitope is optionally polyclonal or monoclonal. An intact antibody, a fragment thereof (e.g., Fab or F(ab′)₂), or an engineered variant thereof (e.g., sFv) can also be used. Such antibodies can be of any immunoglobulin class including IgG, IgM, IgE, IgA, IgD and any subclass thereof. Methods of producing antibodies directed to specific sequences of protein or nucleic acid are known in the art. Optional methods of producing and screening for antibodies that will act as an agent are described by Birch, J R, and Racher, A J, Advanced Drug Delivery Reviews, 2006; 58:671-685.

The antibodies useful in the processes can be prepared by generating B cell hybridomas, as is known in the art, or by using laboratory animals such as mouse, humanized mouse, rat, rabbit or goat which are immunized with the RABV glycoprotein. Briefly, animals such as mice or rabbits are inoculated with the immunogen in adjuvant, and spleen cells are harvested and mixed with a myeloma cell line. The cells are induced to fuse by the addition of polyethylene glycol. Hybridomas are chemically selected by plating the cells in a selection medium containing hypoxanthine, aminopterin and thymidine (HAT). Hybridomas are subsequently screened for the ability to produce monoclonal antibodies that bind either a linear or conformational epitope in RABV glycoprotein. Hybridomas producing antibodies are cloned, expanded and stored frozen for future production.

An antibody or RABV glycoprotein is optionally purified. The term “purified” or “isolated” as used herein, is intended to refer to a composition, isolatable from other components, wherein the protein or peptide is purified to any degree relative to its naturally-obtainable state, i.e., relative to its purity within a cell, relative to is purity within a virion, or relative to its purity within an infective organism. An isolated nucleic acid, protein, or peptide also refers to a nucleic acid, protein or peptide, free from the environment in which it may naturally occur.

Generally, “purified” or “isolated” will refer to a protein or peptide composition that has been subjected to fractionation to remove various other components, and which composition substantially retains its expressed biological activity, if any. Where the term “substantially” purified is used, this designation will refer to a composition in which the protein or peptide forms the major component of the composition, such as constituting about 50% or more of the nucleic acids or proteins in the composition.

Various methods for quantifying the degree of purification of the protein or peptide will be known to those of skill in the art. These include, for example, determining the specific activity of an active fraction, or assessing the number of polypeptides within a fraction by SDS/PAGE analysis. An optional method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the protein or peptide exhibits a detectable activity.

Various techniques suitable for use in protein purification will be well known to those of skill in the art. These include, for example: precipitation with ammonium sulphate, polyethylene glycol, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in its most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of product, or in maintaining the activity of an expressed protein. A protein is optionally substantially purified to a purity of 70%, 75%, 80%, 85%, 90%, 91%, 92%, 93%, 94%, 95%, 96%, 97%, 98%, 99%, or more, or any value or range therebetween.

It is known that the migration of a polypeptide can vary, sometimes significantly, with different conditions of SDS/PAGE (Capaldi et al., Biochem. Biophys. Res. Comm., 76:425, 1977). It will, therefore, be appreciated that under differing electrophoresis conditions, the apparent molecular weights of purified or partially purified expression products may vary.

Vaccines and methods for their use to induce active immunity and protection against lyssavirus induced illness in a subject are provided according to the present invention whereby an inventive process detects the presence or absence of sufficient immunogenicity at a particular concentration of immunogen. The an inventive process can provide a suitable vaccine or immunogen and confidence that the vaccine or immunogen will be functional optionally without the need to resort to difficult and time consuming in vivo assays, or supplementing such assays. The processes provided are useful as a screening assay for new vaccine candidates in a vaccine development program, or for use in quality control procedures during or following vaccine production.

The term “vaccine composition” is used herein to refers to a composition including a biological agent (e.g. protein or peptide) capable of inducing an immune response in a subject inoculated with the vaccine composition. In particular embodiments, the biological agent is a live attenuated and/or inactive RABV. In further embodiments, the biological agent is an antigenic portion or the entire sequence of a RABV glycoprotein.

In particular embodiments of the invention, a positive indication of sufficient immunogenicity is obtained from a process. Optionally, a positive indication is an ECL value of 10,000 counts or greater in a sample that is 10 μg/ml of protein concentration, optionally as derived from a BCA assay or Bradford assay, or optionally a similar ratio of counts/concentration such as 1,000 counts/μg in a 1 ml sample. In other embodiments, a positive indication of antigenicity is an ECL value of 4400 counts or greater in a sample that is 10 μg/ml of protein concentration, optionally as derived from a BCA assay or Bradford assay, or optionally a similar ratio of counts/concentration such as 440 counts/(μg/ml). A positive indication of sufficient immunogenicity is optionally used as a basis for producing a vaccine based on the glycoprotein antigen used in the inventive assay. A positive antigen for inclusion in a vaccine composition of the present invention is prepared by standard methods typically used for preparation of live or inactivated RABV. For example, generally a compatible cell type is inoculated with a RABV and the cells are maintained under conditions that allow for viral replication and production of infectious particles.

RABV proteins such as the RABV glycoprotein are optionally modified to increase their immunogenicity. In a non-limiting example, the antigen may be coupled to chemical compounds or immunogenic carriers, provided that the coupling does not interfere with the desired biological activity of either the antigen or the carrier. For a review of some general considerations in coupling strategies, see Antibodies, A Laboratory Manual, Cold Spring Harbor Laboratory, ed. E. Harlow and D. Lane (1988). Useful immunogenic carriers known in the art include, without limitation: keyhole limpet hemocyanin (KLH); bovine serum albumin (BSA); ovalbumin; PPD (purified protein derivative of tuberculin); red blood cells; tetanus toxoid; cholera toxoid; agarose beads; activated carbon; or bentonite. Useful chemical compounds for coupling include, without limitation, dinitrophenol groups and arsonilic acid.

RABV particles or glycoproteins are harvested, optionally from cell culture supernatant for inclusion in a vaccine composition. The RABV particles or glycoproteins may be isolated from the cell culture supernatant, for example by filtration and/or centrifugation. The isolated RABV particles or glycoproteins are optionally lyophilized, such as for later resuspension in a pharmaceutically acceptable carrier.

The term “pharmaceutically acceptable carrier” refers to a carrier which is substantially non-toxic to a subject and substantially inert to the lyssavirus or portion thereof included in a vaccine composition. A pharmaceutically acceptable carrier is a solid, liquid or gel in form and is typically sterile and pyrogen free.

A vaccine composition may be in any form suitable for administration to a subject.

A vaccine composition is administered by any suitable route of administration including oral and parenteral such as intravenous, intradermal, intramuscular, mucosal, nasal, or subcutaneous routes of administration.

For example, a vaccine composition for parenteral administration may be formulated as an injectable liquid including a RABV, RABV protein or nucleic acid, or fragment thereof, and a pharmaceutically acceptable carrier. Examples of suitable aqueous and nonaqueous carriers include water, ethanol, polyols such as propylene glycol, polyethylene glycol, glycerol, and the like, suitable mixtures thereof; vegetable oils such as olive oil; and injectable organic esters such as ethyloleate. Proper fluidity can be maintained, for example, by the use of a coating such as lecithin, by the maintenance of a desirable particle size in the case of dispersions, and/or by the use of a surfactant, such as sodium lauryl sulfate. A stabilizer is optionally included such as, for example, sucrose. EDTA, EGTA, and an antioxidant.

Detailed information concerning customary ingredients, equipment and processes for preparing dosage forms is found in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004, throughout and in chapter 16; A. R. Gennaro, Remington: The Science and Practice of Pharmacy, Lippincott Williams & Wilkins, 21^{st} ed., 2005, particularly chapter 89; and J. G. Hardman et al., Goodman & Gilman's The Pharmacological Basis of Therapeutics, McGraw-Hill Professional, 10th ed., 2001.

An adjuvant is optionally included in a virus composition according to embodiments of the present invention. Adjuvants are known in the art and illustratively include Freund's adjuvant, aluminum hydroxide, aluminum phosphate, aluminum oxide, saponin, dextrans such as DEAE-dextran, vegetable oils such as peanut oil, olive oil, and/or vitamin E acetate, mineral oil, bacterial lipopolysaccharides, peptidoglycans, and proteoglycans. An immunoenhancing tag is optionally an adjuvant.

The term “subject” is used herein to refer to a human, non-human animals, illustratively including other primates, cows, horses, sheep, goats, pigs, dogs, cats, birds, poultry, and rodents such as mice or rats.

Viral proteins of the present invention may also be used in the form of pharmaceutically acceptable salts. Suitable acids and bases which are capable of forming salts with the proteins of the present invention are well known to those of skill in the art, and include inorganic and organic acids and bases.

Optionally, vaccine or glycoprotein immunogen may also contain conventional pharmaceutical ingredients, such as preservatives, or chemical stabilizers. Suitable ingredients operable herein include, for example, casamino acids, sucrose, gelatin, phenol red, N—Z amine, monopotassium diphosphate, lactose, lactalbumin hydrolysate, and dried milk.

The phrase “therapeutically effective amount” is used herein to refer to an amount effective to induce an immunological response sufficient to prevent or ameliorate signs or symptoms of a lyssavirus-mediated disease. Induction of an immunological response in a subject can be determined by any of various techniques known in the art, illustratively including detection of anti-lyssavirus antibodies, measurement of anti-lyssavirus antibody titer and/or lymphocyte proliferation assay. Illustrative methods for detection of anti-lyssavirus antibodies are illustrated by Hanlon, C A., et al., Virus Res., 2005; 111(1):44-54. Signs and symptoms of lyssavirus-mediated disease may be monitored to detect induction of an immunological response to administration of a vaccine composition of the present invention in a subject. An immunological response is illustratively a reduction of clinical signs and symptoms of lyssavirus-mediated disease. An immunological response is illustratively, development of anti-lyssavirus antibodies, activation of T-cells, B-cells, or other immune cells following administration of an inventive composition, or other immune responses known in the art.

In some embodiments, a therapeutically effective amount is determined at least in part by the ECL assay of the invention. An amount or glycoprotein that produces a positive immunogenicity signal, optionally 10,000 ECL counts, optionally 4400 ECL counts, for a 10 μg/ml sample or optionally a ratio equivalent, will determine whether the amount of glycoprotein is a therapeutically effective amount.

In some embodiments, a method of inducing an immunological response against a lyssavirus-mediated disease in a subject includes administering 10⁴ to 10⁸ ffu of live attenuated vaccine or 1 to 25 micrograms of inactivated virus in a typical vaccine composition.

Administration of a vaccine composition with sufficient immunogenicity as determined by the inventive ECL assay includes administration of one or more doses of a vaccine composition to a subject at one time in particular embodiments. Alternatively, two or more doses of a vaccine composition are administered at time intervals of days, weeks, or years. A suitable schedule for administration of vaccine composition doses depends on several factors including age and health status of the subject, type of vaccine composition used and route of administration, for example. One of skill in the art is able to readily determine a dose and schedule of administration to be administered to a particular subject.

A biological sample is any sample derived from a biological source including an animal, plant, tissue, or cell. A biological sample is illustratively tissue derived from brain such as the brain stem or cerebellum, other neuronal tissue, kidney, heart, or other tissue. In some embodiments, a biological sample is blood, plasma, serum, urine, feces, saliva, nasal secretions, lung aspirate, cerebrospinal fluid, or skin.

Various aspects of the present invention are illustrated by the following non-limiting examples. The examples are for illustrative purposes and are not a limitation on any practice of the present invention. It will be understood that variations and modifications can be made without departing from the spirit and scope of the invention.

### EXAMPLES

### Example 1

Monoclonal antibodies (mAb) against RABV G were purified from existing hybridomas. CDC mAb 2-21-14 was generated in two cloning steps from B-cells isolated from a BALB/c mouse immunized with Ethiopian street RABV fused with Sp2/0-Ag 14 derivative of the BALB/c myeloma line P3-X63-AG8 in 1984. CDC mAbs 62-80-6 and 62-71-3 were generated in two cloning steps from B-cells isolated from a BALB/c mouse immunized with RABV ERA strain fused with Sp2/0-Ag 14 derivative of the BALB/c myeloma line P3-X63-AG8 in 1982. CDC mAbs 2-21-14 and 62-80-6 are isotype IgG2a and IgG2b, and CDC mAb 62-71-3 is isotype IgG3.

Using a direct ECL assay, MAb 2-21-14 could bind to RABV antigens in the native form but could not bind to heat denatured antigens. MAb 62-80-2 could bind to RABV antigens in the native or heat denatured form. CDC mAbs 62-80-6 and 62-71-3 bind to linear epitopes while mAb 2-21-14 binds a conformational epitope.

The epitope for each MAb was determined by selecting escape mutants according to the method of Marissen, et al. [24]. The coding region of the G gene was sequenced following Ellison, et al., Zoonoses Public Health, 2013; 60(1):46-57. The binding epitope for MAb 62-80-6 was mapped to antigenic site I by sequencing escape mutants. Attempts to isolate escape mutants of MAb 2-21-14 were not successful. However, analysis of natural escape RABV variants from red bats (Lasiurus borealis) and hoary bats (Lasiurus cinereus) suggests binding to antigenic site III.

Labeled antibodies are prepared using Zeba Spin Desalting Columns, 7K MWCO (Thermo Scientific, Rockford, Ill.) equilibrated with 0.01M PBS pH 7.9 according to manufacturer's instructions before eluting the secondary mAb. The protein concentration was adjusted to 2 mg/ml using the same buffer, and the antibody was conjugated with 3 nmol/μl SULFO-TAG NHS-Ester (Meso Scale Discovery, Gaithersburg, Md.) according to manufacturer's instructions for 2 h at room temperature in the dark with shaking. Excess SULFO-TAG NHS-Ester was removed using the Zeba Spin Desalting Columns following Meso Scale Discovery's instructions. Protein concentration of the conjugated antibody was determined using the BCA Protein Assay (Thermo Scientific, Rockford, Ill.) according to manufacturer's instructions.

### Example 2

The amount of glycoprotein antigenicity is measured by a capture ECL assay using the antibodies of Example 1 using either a direct measurement method or a capture method. For a direct method, standard, bare 96-well carbon-electrode plates (MSD, Gaithersburg, Md., USA) were coated with 10 μg/ml of antigen from various commercial vaccines either in the native form or heat denatured and incubated overnight at 4° C. Plates were washed three times with 0.01M PBS-0.05% Tween20 (Sigma-Aldrich, St. Louis, Mo., USA), 150 μl/well of casein (Vector Laboratories, Burlingame, Calif., USA) was added, and plates were incubated 60 min at room temperature with shaking. Casein was removed, and 1 μg/ml conjugated MAb of Example 1 was added to the wells. Plates were incubated 60 m at room temperature with shaking, then washed three times with 0.01M PBS-0.05% Tween20, and 150 μl/well of Reading Buffer T (MSD, Gaithersburg, Md., USA) was added. The ECL values for each well were measured using a SECTOR Imager 6000 (MSD, Gaithersburg, Md., USA).

For capture methods, standard, bare 96-well carbon-electrode plates from Meso Scale Discovery (Gaithersburg, Md.) were coated with 1 μg/ml of primary mAb diluted in 0.01M PBS pH 7.4-7.6 and incubated overnight at 4° C. Plates were washed three times with 0.01M PBS-0.05% Tween20 (Sigma-Aldrich, St. Louis, Mo.), 150 μl/well of casein (Vector Laboratories, Burlingame, Calif.) was added, and plates were incubated 60 m at room temperature with shaking. Casein was removed, antigens were added with a minimum 24 μl/well, and plates were incubated 60 m at room temperature with shaking. Plates were washed three times with 0.01M PBS-0.05% Tween20, 30 μl/well of 1 μg/ml conjugated secondary mAb 62-80-6-sulfo was added, and plates were incubated 60 m at room temperature with shaking. Plates were washed three times with 0.01M PBS-0.05% Tween20, 150 l/well of Reading Buffer T (Meso Scale Discovery, Gaithersburg, Md.) was added, and ECL values for each well were measured using a SECTOR Imager 6000 (Meso Scale Discovery, Gaithersburg, Md.).

A gradient from 0.5 μg/ml to 4 μg/ml of both primary and secondary antibody was used to determine the best antibody concentrations for the ECL assay. When using MAb 2-21-14 for both capture and detection, the highest signal to background ratio was at a low primary MAb concentration and high secondary MAb concentration. The opposite was true when using MAb 62-80-6 for detection: the highest signal to background ratio was at a high primary MAb concentration and low secondary MAb concentration. Using 0.5 μg/ml of MAb 2-21-14 for capture and 1 μg/ml of MAb 2-21-14 for detection resulted in a signal to background ratio of approximately 70, and using 1 μg/ml of MAb 2-21-14 for capture and 0.5 μg/ml of MAb 62-80-6 for detection resulted in a signal to background ratio of approximately 10,000. The difference between MAb 62-80-6 and 2-21-14 may result from differences in the SULFO tag labeling or availability of the binding epitopes.

### Example 3

### Comparison of Purified RABV G Protein and Vaccine Lots by ECL Assay

Commercially available RABV vaccines Imovax (lot: G1076) and RabAvert (lot: 464011A) were purchased from Sanofi Pasteur (Swiftwater, Pa., USA) and Novartis Pharmaceuticals (Dorval, Quebec, Canada), respectively, and reconstituted according to the manufacturer's instructions. Depleted vaccines were generated by reconstituting the same vaccines and incubating for 100 days at 37° C. An experimental RABV vaccine (lot: CMB-0300-007) not commercially available was provided by Fraunhofer (Newark, Del.). Adjuvanted vaccine was generated by mixing the same vaccine 50% (v/v) with Alhydrogel (Accurate Chemical, Westbury, N.Y.). A second experimental RABV vaccine (lot: 475-03-020) was provided by Medicago (Quebec, Quebec, Canada).

Purified RABV G with a sequence found at GenBank accession no: ABX46666 was produced from ERA and CVS-11 RABV strains. These RABV strains were propagated with cultured BHK cells (Invitrogen, Carlsbad, Calif., USA) in 150 cm² flasks (Corning Life Sciences, Tewksbury, Mass., USA) to 1×10⁹ ffu/ml and inactivated with 0.1% (v/v) β-propiolactone (Sigma-Aldrich, St. Louis, Mo., USA) for 5 h at 0° C. The RABV was concentrated from the cell culture supernatant by centrifugation for 2 h at 50,000×g and envelope G was purified as previously described in Dietzschold B. Techniques for the purification of rabies virus, its subunits and recombinant products. In: Meslin F X, Kaplan M M, Koprowski H, editors. Laboratory techniques in rabies. 4th ed. Geneva: World Health Organization, 1996: 175-80. Denatured G was prepared by sonication of concentrated virus during purification, or by heating native purified G for 10 min at 98° C. The total protein concentration of the test vaccines was determined using the BCA Protein Assay (Thermo Scientific, Rockford, Ill., USA) according to manufacturer's instructions.

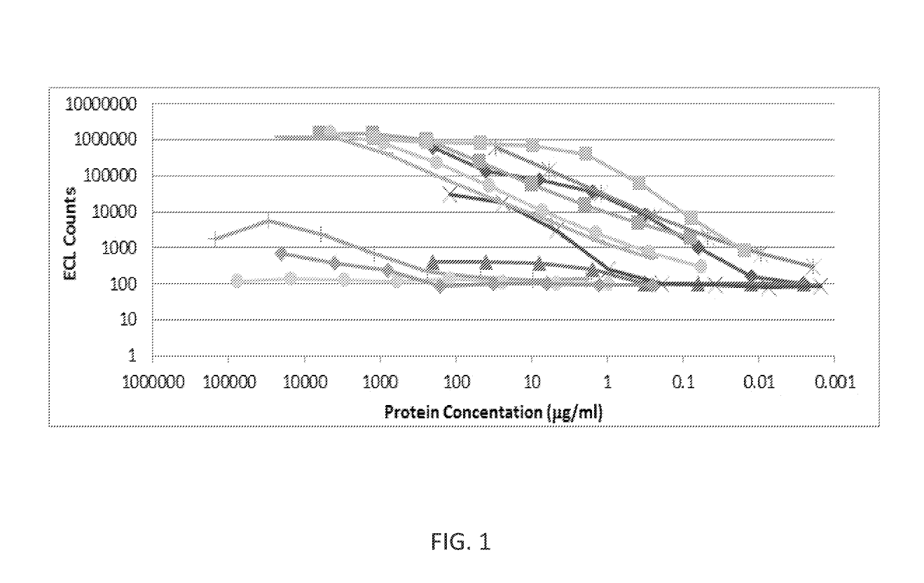

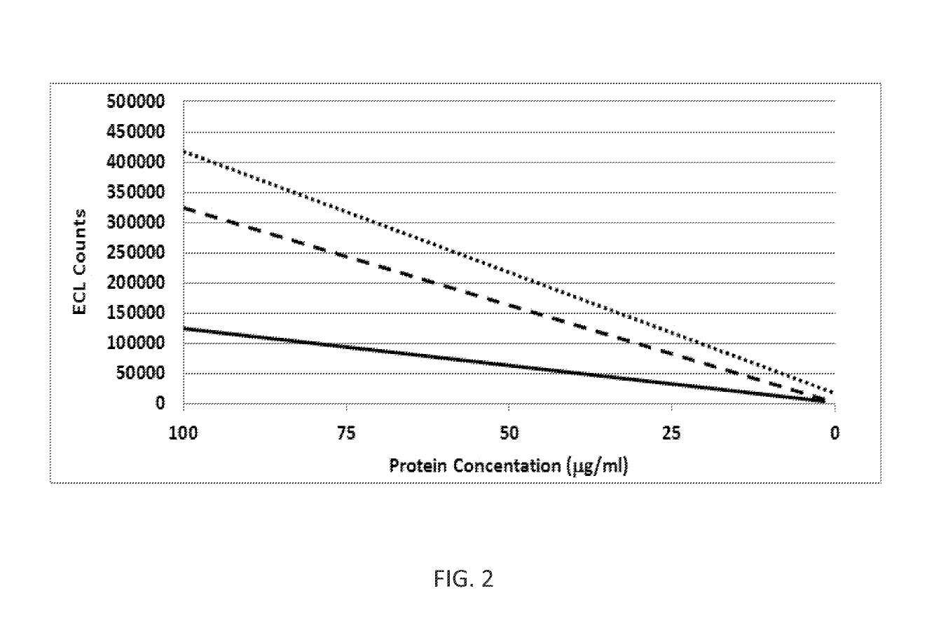

Using the MAb 2-21-14 for antigen capture, the ECL counts of tested vaccines were determined using eight 5-fold dilutions (FIG. 1). The antigen content or antigenicity of the vaccines was defined as the quality and quantity of RABV G detected by the antigen capture ECL assay. Antigenicity levels were based on the linear regression of the ECL counts and total protein concentration and calculated at 25 μg/ml for each vaccine (Table 1, FIG. 2). The purified RABV ERA G (2005) demonstrated the highest calculated antigenicity of the vaccines tested. When RABV ERA G was diluted, the counts actually increased. Purified RABV G was prepared at different times, such that antigenicity increased with each subsequent lot. Purified RABV ERA G (2012) had the next highest antigenicity followed by CVS G (2011) followed by ERA G (2010).

TABLE 1
Calculation of antigen content.

| | Protein concentration | | | | |
|---|---|---|---|---|---|
| Vaccine (lot) | (μg/ml) | m^{a} | b^{b} | y^{c} | Counts*(μg/ml)^{-1d} |
| ERA G (2005) | 1 200 | 1215800 | −9157 | 30385843 | 1 000 000 |
| ERA G (2005) Diluted | 200 | 1397800 | −7185 | 34937815 | 1 000 000 |
| ERA G (2012) | 2 500 | 2949 | 70297 | 144022 | 6 000 |
| ERA G (2012) Diluted | 200 | 2798 | 15637 | 85587 | 3 000 |
| CVS G (2011) | 120 | 253 | 3048 | 9373 | 400 |
| ERA G (2010) | 200 | 31 | 142 | 917 | 40 |
| RabAvert ® (464011A) | 6 000 | 4015 | 14553 | 114928 | 4 000 |
| RabAvert ® Diluted | 2 000 | 3214 | 2576 | 82926 | 3 000 |
| RabAvert ® Depleted | 4 500 | 1217 | 2702 | 33127 | 1 000 |
| Imovax ® (G1076) | 20 000 | 473 | 4742 | 16567 | 700 |
| Imovax ® Depleted | 20 000 | 0.023 | 247 | 248 | 10 |
| Fraunhofer ® | 150 000 | 0.177 | 606 | 610 | 20 |
| Fraunhofer ® Adjuvant | 75 000 | 0.001 | 119 | 119 | 5 |
| Medicago ® | 150 | 20482 | 7320 | 519370 | 20 000 |

^{a}Slope of the linear regression based on average ECL counts for eight 5-fold dilutions plotted against protein concentration
^{b}y-intercept of the linear regression
^{c}ECL counts (y) at 25 μg/ml (x) based on the linear regression
^{d}ECL counts divided by 25 μg/ml

Commercially available vaccines were tested by the same procedures. Both Imovax® and RabAvert® demonstrated similar total ECL counts. However, RabAvert® had a lower protein concentration resulting in higher counts/(g/ml). As expected, when RabAvert® was diluted the antigenicity decreased. Depletion of these vaccines was attempted by incubating at increased temperature. While the antigen content of both depleted vaccines decreased, the antigenicity of RabAvert® remained high, but that of Imovax® was diminished (Table 1).

An experimental rabies vaccine produced by Fraunhofer® had a very high total protein concentration but low antigen content. When the same vaccine was mixed with adjuvant (Fraunhofer® adjuvant), both the total protein concentration and antigenicity were reduced. Fraunhofer® with adjuvant had the lowest counts μg⁻¹ ml⁻¹ of any vaccine tested. A second experimental rabies vaccine produced by Medicago® had a low total protein concentration but high antigenicity (Table 1).

The results of these studies indicate that in the inventive ECL assay using the exemplary antibodies, a vaccine is considered immunogenic if it induced a rVNA titer >0.5 IU/ml. Imovax® had the lowest antigenicity at 700 counts μg⁻¹ ml⁻¹ of the vaccines that were considered immunogenic by these standards, and CVS G (2011) had the highest antigenicity at 400 counts μg⁻¹ ml⁻¹ of the vaccines that were not immunogenic. The statistical cut-off of 440 counts/(μg/ml) falls within this empirical range. Using MAb 2-21-14 resulted in 100% concordance in this preliminary analysis, and borderline vaccine lots were accurately classified.

### Example 4

### Comparison of ECL Assay to In Vivo Vaccination Results

As an immunogenicity comparator, the immunogenicity of various vaccine preparations are determined in animals. An approved animal use protocol was established with CDC Institutional Animal Care and Use Committee (protocol #2332SMIMOUC-A3). Female, 4-week-old, CD-1 mice were purchased from Charles River Laboratory (Wilmington, Mass.). Mice were not used for any experiments prior to the recommended quarantine and acclimation period. For each vaccine tested, mice were divided into groups with 10 mice in each group. On day 0, mice were vaccinated intramuscularly with 50 μl of VLP in the quadriceps muscle. The immune response was assessed in all animals by taking approximately 200 μl of blood from the submandibular region using a Goldenrod Lancet (MEDIpoint, Mineola, N.Y.) on days 0, 14, and 30 for determination of rabies virus specific VNA titer using a rapid focus-forming inhibition test (RFFIT) or a modified RFFIT for small volumes of serum (5). All animals were challenged intramuscularly in the quadriceps muscle with 50 μl of street canine rabies virus (RV3374R) on day 30. All intramuscular injections were done using a tuberculin syringe with pre-measured inoculum and 32 gauge needle. Starting on the day of challenge mice were observed once a day by investigators and from 7 to 21 days after challenge mice were observed twice daily by investigators for disease or death. Starting on day 22 after challenge until the experimental endpoint mice were observed once a day by investigators. Mice were euthanized by carbon dioxide asphyxiation at the first clinical signs of rabies (e.g. paresis, paralysis, aggression), according to approved euthanasia criteria, or as directed by the attending veterinarian. Cervical dislocation was used as a secondary mode of euthanasia. During necropsy, the brain stem was harvested to confirm rabies diagnosis by the direct fluorescent antibody test (2). All mice surviving up to 45 days after the challenge were euthanized, and selected animals were necropsied for rabies diagnosis. The results of the vaccine tests are illustrated in Table 2.

TABLE 2
Pre-exposure Vaccination in Mice

| | Dose | | | | Survi- | |
|---|---|---|---|---|---|---|
| Vaccine (lot) | (total protein) | | rVNA | | vorship | |
| ERA G (2005) Diluted | 10 | μg | 4.2 | IU/ml | 10/10 | (100%) |
| ERA G (2010) | 10 | μg | 0.17 | IU/ml | 6/10 | (60%) |
| CVS G (2011) | 6 | μg | 0.07 | IU/ml | 7/9 | (77%) |
| Imovax ® (G1076) | 1000 | μg | 2.9 | IU/ml | 10/10 | (100%) |
| Imovax ® Depleted | 1000 | μg | <0.05 | IU/ml | 7/10 | (70%) |
| RabAvert ® (464011A) | 100 | μg | 0.6 | IU/ml | 9/9 | (100%) |
| Diluted | | | | | | |
| RabAvert ® Depleted | 225 | μg | 4.7 | IU/ml | 10/10 | (100%) |
| Fraunhofer ® | 7500 | μg | 0.06 | IU/ml | 5/9 | (55%) |
| (0300-007) | | | | | | |
| Fraunhofer ® Diluted | 1000 | μg | <0.05 | IU/ml | 1/8 | (12%) |
| Fraunhofer ® Adjuvant | 3750 | μg | 0.07 | IU/ml | 7/10 | (70%) |

| | | | | | |
|---|---|---|---|---|---|
| PBS Negative Control | ND | <0.05 | IU/ml | 3/16 | (19%) |

Of the vaccines tested, ERA G (2005 lot diluted), Imovax®, RabAvert® and RabAvert® depleted induced VNA titers >0.5 IU/ml and protected 100% of mice from peripheral RABV challenge. ERA G (2010), CVS G, Imovax® depleted, Fraunhofer®, and Fraunhofer® with adjuvant induced VNA titers <0.5 IU/ml and provided disparate levels of protection.

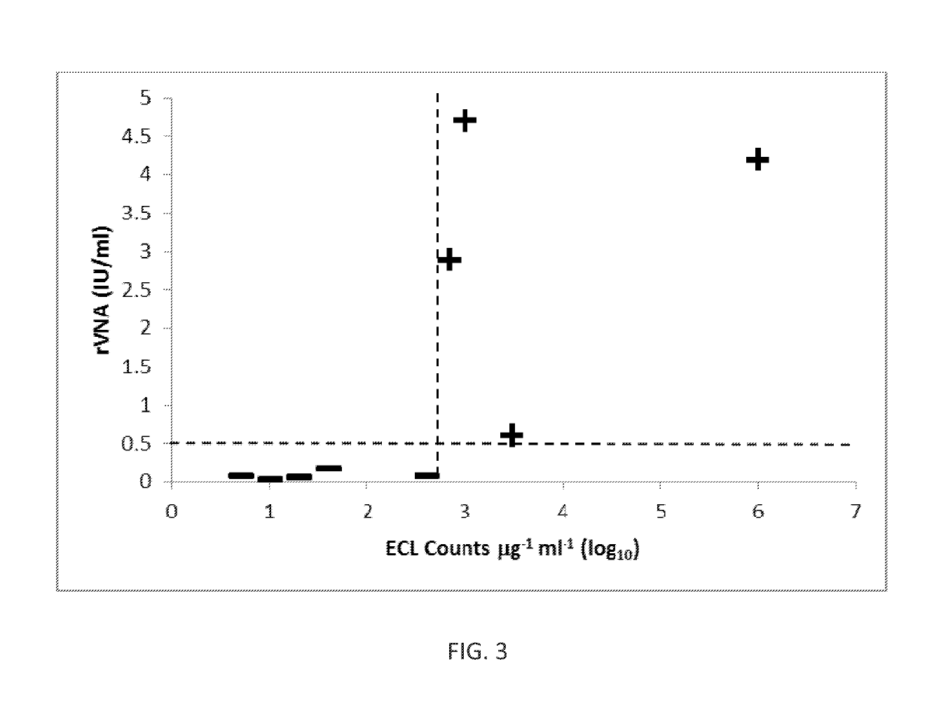

The ECL assay using MAb 2-21-14 as in Example 3 was able to accurately distinguish vaccines that induced VNA titer >0.5 IU/ml from vaccines that induced VNA titer <0.5 IU/ml (FIG. 3). A weak correlation, R²=0.51, existed between the log transformed antigenicity values and VNA titers. The statistical cut-off, calculated from the mean antigenicity of non-immunogenic vaccines plus two standard deviations, was 440 counts μg⁻¹ ml⁻¹.

Overall, the greater the level of glycoprotein of native conformation correlates with increased immunogenicity as measured by both anti-glycoprotein antibody titers and mouse survival at 75 days post challenge.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

### REFERENCE LIST

1. Barth, R., G. Diderrich, and E. Weinmann. 1988. NIH test, a problematic method for testing potency of inactivated rabies vaccine. Vaccine 6:369-77.
2. Dean, D. J., M. K. Abelseth, and P. Atanasiu. 1996. The fluorescent antibody test, p. 88-95. In F. X. Meslin, M. M. Kaplan, and H. Koprowski (ed.), Laboratory techniques in rabies, 4th ed. World Health Organization, Geneva.
3. Dietzschold, B. 1996. Techniques for the purification of rabies virus, its subunits and recombinant products, p. 175-180. In F. X. Meslin, M. M. Kaplan, and H. Koprowski (ed.), Laboratory techniques in rabies, 4th ed. World Health Organization, Geneva.
4. Jivapaisarnpong, T., T. Schofield, and P. R. Krause. 2009. A vaccine measured with a highly variable assay: rabies. Biologicals 37:412-5; discussion 421-3.
5. Smith, J. S., P. A. Yager, and G. M. Baer. 1973. A rapid reproducible test for determining rabies neutralizing antibody. Bull World Health Organ 48:535-41.

Various modifications of the present invention, in addition to those shown and described herein, will be apparent to those skilled in the art of the above description. Such modifications are also intended to fall within the scope of the appended claims.

Patents and publications mentioned in the specification are indicative of the levels of those skilled in the art to which the invention pertains. These patents and publications are incorporated herein by reference to the same extent as if each individual application or publication was specifically and individually incorporated herein by reference for the material for which it is cited as well as all other teaching contained therein.

The foregoing description is illustrative of particular embodiments of the invention, but is not meant to be a limitation upon the practice thereof. The following claims, including all equivalents thereof, are intended to define the scope of the invention.

We claim:

1. A process of determining immunogenicity of a rabies virus vaccine comprising:
interacting a test RABV glycoprotein with a first anti-glycoprotein antibody;
binding said glycoprotein with a second anti-glycoprotein antibody simultaneous with said first anti-glycoprotein antibody, said second glycoprotein antibody comprising an electrochemiluminescent marker; and
detecting the magnitude of signal produced by said second anti-glycoprotein antibody using said marker.

2. The process of claim 1 wherein said first anti-glycoprotein antibody is a conformationally specific antibody that binds an epitope in antigenic site III.

3. The process of claim 1 further comprising determining the immunogenicity of said test RABV glycoprotein as a rabies virus vaccine by said step of detecting.

4. The process of claim 1 further comprising producing said RABV glycoprotein as a vaccine or rejecting said RABV glycoprotein for production.

5. The process of claim 1 wherein said signal is compared to a glycoprotein potency standard curve established by interacting a plurality of known standard RABV glycoprotein concentrations with an anti-glycoprotein antibody indistinguishable from said first anti-glycoprotein antibody;
binding said standard glycoprotein with a detection anti-glycoprotein antibody indistinguishable from said second anti-glycoprotein antibody;
detecting the quantity of said standard glycoprotein using said marker for each concentration to produce a standard signal; and
constructing said standard curve using said signals.

6. The process of claim 1 wherein said electrochemiluminescent marker comprises a SULFO-TAG NHS-ester.

7. The process of claim 1 further comprising comparing the quantity of said glycoprotein to a glycoprotein immunogenicity standard.

8. The process of claim 1 wherein said second anti-glycoprotein antibody binds a linear epitope on said RABV glycoprotein.

9. The process of claim 4 wherein said step of producing said vaccine is performed when the ECL value of said signal is or exceeds 4,400 in a sample with a protein concentration of 10 micrograms per milliliter.

10. The process of claim 1 further comprising administering a therapeutically effective amount of a vaccine to a subject, said vaccine comprising a glycoprotein determined to have sufficient immunogenicity.

11. The process of claim 10 wherein said therapeutically effective amount is determined by said step of detecting.

12. The process of claim 1 wherein said glycoprotein is unlabeled or absent an immunoenhancing tag.

13. The process of claim 1 wherein said first anti-glycoprotein antibody and said second anti-glycoprotein antibody are compositionally identical.

14. The process of claim 1 wherein said first anti-glycoprotein antibody and said second anti-glycoprotein antibody are both conformationally specific antibodies.

15-21. (canceled)

22. A process of determining the immunogenicity of a rabies virus vaccine comprising:
interacting a test RABV glycoprotein with antibody 2-21-14;
binding said glycoprotein with a second anti-glycoprotein antibody simultaneous with said first anti-glycoprotein antibody, said second glycoprotein antibody comprising an electrochemiluminescent marker; and
detecting the magnitude of signal produced by said second anti-glycoprotein antibody using said marker.

23-28. (canceled)

29. A process of producing a rabies virus vaccine comprising:
interacting a test RABV glycoprotein with a first conformationally specific anti-glycoprotein antibody;
binding said test RABV glycoprotein with a second anti-glycoprotein antibody simultaneous with said first anti-glycoprotein antibody, said second glycoprotein antibody comprising an electrochemiluminescent marker;
detecting the magnitude of signal produced by said second anti-glycoprotein antibody using said marker; and
producing said vaccine when said signal is or exceeds 4,400 in a sample with a protein concentration of 10 micrograms per milliliter.

30. The process of claim 29 wherein said second anti-glycoprotein antibody is antibody 2-21-14, antibody 62-80-6, or antibody 62-71-3.

31. The process of claim 29 wherein said wherein said electrochemiluminescent marker comprises a SULFO-TAG NHS-ester.

32. The process of claim 29 further comprising administering a therapeutically effective amount of said vaccine to a subject.

33. The process of claim 29 further comprising administering a therapeutically effective amount of sai [symbol]
